(19) United States
(12) Patent Application Publication
(10) Pub. No.: US 2010/0185043 A1
(43) Pub. Date: Jul. 22, 2010

(54) HEART ASSIST DEVICE, CANNULA AND FILTER THEREFOR

(75) Inventors: John Campbell Woodard, Turramurra (AU); Genevieve Christine O'shea, Pittsburgh, PA (US)

Correspondence Address: DANIEL B. SCHEIN, PH.D., ESQ., INC., P. O. BOX 68128, Virginia Beach, VA, 23471, US

(73) Assignee: VENTRASSIST PTY LTD, Brisbane, QL (AU)

(21) Appl. No.: 12/532,150

(22) PCT Filed: Mar. 18, 2008
(86) PCT No.: PCT/AU08/00385

§ 371 Date: Mar. 29, 2010

(30) Foreign Application Priority Data
Mar. 20, 2007 (AU) 2007901448

(51) Int. Cl. A61M 1/12

(52) U.S. Cl. 600/16

(57) ABSTRACT

A cannula, such as an implantable cannula, for an implantable blood pump is disclosed. The cannula comprises a body having a bore extending between an inlet and an outlet for fluid communication therebetween. A filter is disposed in, on or at the body and arranged to filter blood clots from at least a portion of fluid passing through the body, and therefore into the blood pump. The cannula is preferably arranged to be mounted between a patient's left ventricle and an implantable blood pump. Also disclosed is a filter and a heart assist device, such as a blood pump, incorporating a filter to prevent blood clots from entering the blood pump.

### TECHNICAL FIELD

The present invention relates to heart assist devices, cannulae and filters therefor, and in particular to implantable blood pumps, such as ventricular assist devices.

### BACKGROUND ART

Thrombogenesis is considered a potential issue heart assist devices including, but not limited to, extracorporeal cardiac bypass machines and implantable blood pumps such as left ventricular assist devices.

Cannulation is normally required to fluidly connect the heart assist device to the body's blood flow system, sometimes directly connecting the heart assist device to the heart. Some known outflow cannulae include filters to remove blood clots from the blood flow. For example, cardiac bypass machines have traditionally included a filter on outflow cannulation. These filters are generally limited to ameliorating blood clots, prior to the blood flow entering the patient's body, generated during processing by the pump of the bypass machine.

Some newer heart assist devices include low thrombogenic technology, such as the implantable blood pump described in U.S. Pat. No. 6,227,797. U.S. Pat. No. 6,227,797 describes a centrifugal-type flow pump incorporating a hydro-dynamic thrust bearing to reduce thrombogenesis. This type of pump produces very low haemolysis and low thrombogenesis compared with other known blood pumps, and as such has generally removed the need for filters to be mounted in their corresponding outflow cannulae as this new pump technology no longer generates significant amounts of blood clotting.

However, the inventors have found that such newer styles of blood pumps may be generally more susceptible to interference by blood clots interfering with the pumping mechanism than the earlier style of blood pumps, such as peristaltic or pulsatile blood pumps.

It is an object of at least one of the embodiments of the present invention to overcome or at least to ameliorate one or more of the disadvantages associated with the above mentioned prior art.

### SUMMARY OF THE INVENTION

According to a first aspect there is provided a cannula for an implantable blood pump, the cannula comprising:

a body having a bore extending between an inlet and an outlet for fluid communication therebetween; and

a filter is disposed in, on or at the body and arranged to filter blood clots from at least a portion of fluid passing through the body.

Advantageously, the use of a filter helps to avoid or reduce the risk of blood clots entering the blood pump. The ability to reduce the risk of thrombus in relation to the pump is desired, as thrombus in the pump may detrimentally alter its desired operation parameters. This may particularly be the case with regard to blood pumps using rotatable impellers for blood flow, such blood pumps which use rotatable impellers for axial or centrifugal blood flow through the pump.

Optionally, the cannula is an implantable inflow cannula.

Optionally, the body is tubular. The body may also be flexible.

Optionally, the cannula is arranged to be mounted between a patient's left ventricle and an implantable blood pump. The outlet of the cannula may be mountable to an inlet of the pump.

The filter may be located at or adjacent the inlet. The filter may be located between the inlet & the outlet or at the outlet.

The filter may be located in or on the body such that all the fluid passing through the body will pass through the filter. Alternatively, the filter may be annular defining an outer screening portion and a central non-screening aperture and is located in or on the body such that some of the fluid passing through the body will pass through the outer screening portion and some of the fluid will pass through the central aperture.

The filter is optionally constructed of a mesh. For example, the mesh may have a three-dimensional porous-type structure, or a two-dimensional matrix-type structure. Alternatively, the filter may have a two or three-dimensional woven mesh type structure. The filter may be stiff and/or flat and/or planar.

Optionally, the filter is constructed of titanium alloy.

The filter may be coated with a biocompatible substrate. The substrate may carry a charge appropriate to attract and capture clots.

Optionally, the body is constructed of silicone.

Optionally, the inlet may be configured to be inserted within the left ventricle of the patient such that the filter is located on the inlet within the left ventricle.

Optionally, the first end comprises a funnel shaped tip.

According to another aspect, there is provided a heart assist device comprising:

a blood pump;

a blood inlet and a blood outlet;

a blood passage from the inlet to the outlet through the pump; and

a filter in cooperation with the blood pump arranged to filter at least a portion of blood passing through the blood pump.

Optionally, the filter is located upstream of the pump. The filter may be arranged to filter all the blood passing through the pump.

The filter may comprise a titanium alloy mesh or matrix and/or may be coated with a biocompatible substrate. The substrate may carry a charge appropriate to attract and capture clots.

The device may comprise an inflow cannula in fluid communication with the blood inlet. The filter may be located in the cannula, for example, it may be located at or adjacent an inlet end of the cannula.

Optionally, the cannula has two ends, a first said end being connectable to the blood inlet and a second said end being mountable to a patient's left ventricle.

Alternatively, the filter may be located adjacent an inlet of the device.

Optionally, the device is a ventricular assist device.

Optionally, the heart assist device comprises a cannula according to the above described first cannula aspect.

According to another aspect, there is provided a heart assist device filter for filtering blood passing through the device, the device comprising a blood pump having a blood inlet and a blood outlet and a pumping means for pumping blood through the device, wherein the filter is positionable upstream of the pumping means to filter at least a portion of blood entering the device.

According to an alternative arrangement, there is provided an inflow cannula for an implanted blood pump, said cannula including a tubular body having a bore extending between at least first and second ends for fluid communication therebetween, and wherein at least one matrix is disposed within said bore for capturing blood clots.

Preferably, said inflow cannula is mounted between the left ventricle and the implanted blood pump.

Preferably, said matrix is positioned proximal to the first end.

Preferably, said matrix is constructed of a mesh.

Preferably, said mesh is constructed of titanium alloy.

Preferably, said mesh is coated with a biocompatible substrate.

Preferably, said substrate carries a charge appropriate to attract and capture clots.

Preferably, said tubular body is constructed of silicone.

Preferably, said first end is inserted within the left ventricle of the patient and matrix is positioned on the first end within the left ventricle.

Preferably, said first end is a funnel shaped tip.

Preferably, said at least one aperture is disposed in said tubular body near said funnel shaped tip.

### BRIEF DESCRIPTION OF THE DRAWINGS

Preferred embodiments of the invention will now be described, by way of example only, with reference to the accompanying drawings in which:

FIG. 1 is a schematic view of an implantable blood pump in connection with a heart and aorta via inflow cannulae incorporating a preferred embodiment;

FIGS. 2 to 4 are sectioned side elevations of cannulae according to alternative embodiments;

FIGS. 5 to 7 are various views of alternative embodiments of filters; and

FIG. 8 is a schematic view of an implantable blood pump in connection with a heart and aorta via inflow cannulae incorporating an alternative embodiment.

### DETAILED DESCRIPTION OF THE PREFERRED EMBODIMENTS

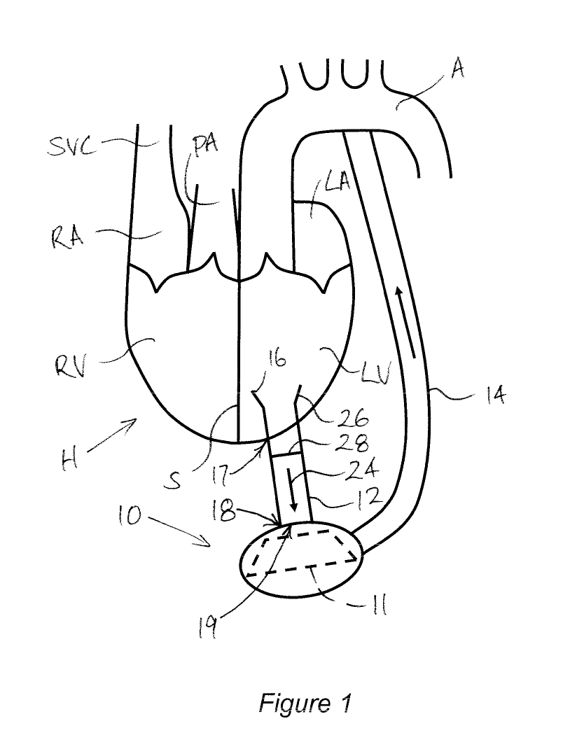

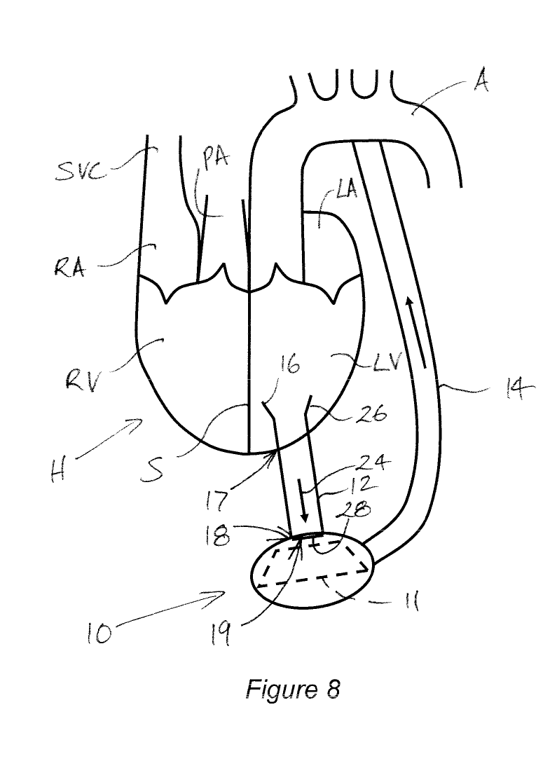

In a first preferred embodiment, as depicted in FIG. 1, an implantable blood pump in the form of a left ventricular assist device (LVAD) 10 is connected to a heart H of a patient to provide assistance to and to reduce loading on the heart H. The LVAD 10 in this embodiment is of the type using known centrifugal flow principles achieved using a rotatable impeller 11. An example of such a blood pump is described in U.S. Pat. No. 6,227,797, which is incorporated herein by reference. The LVAD 10 is connected in parallel to the normal circulation of the heart H. This connection is achieved by the use of an inflow cannula 12 and an outflow cannula 14. FIG. 1 illustrates a simple model of the heart H and part of the circulatory system, comprising left ventricle LV, left atrium LA, right ventricle RV, right atrium RA, aorta A, superior vena cava SVC, pulmonary artery PA and septum S. Also, as will be understood, FIG. 1 (and FIG. 8 described below) are not to scale and provided for illustrative purposes. For instance, the outflow cannula 14 is illustrated on the left hand side of the heart H for convenience of illustration, however in practice it may be positioned elsewhere, such as on the right hand side and/or in front of the heart H.

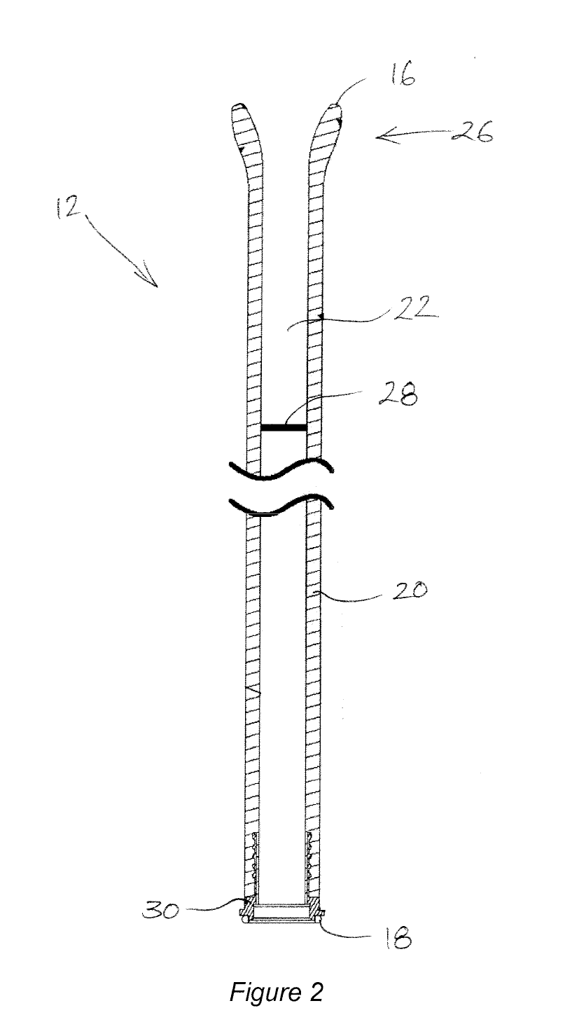

The inflow cannula 12 is connected near an inlet end 16 thereof through an incised hole 17 of the left ventricle LV, such that the inlet end 16 is wholly within the left ventricle LV. Referring to FIG. 2, the inflow cannula 12 is connected at an outlet end 18 thereof to an inlet 19 of the LVAD 10. In this embodiment, the inflow cannula 12 includes an elongate tubular body 20 having a bore 22 extending between the inlet end 16 and the outlet end 28. The bore 22 allows for fluid communication of blood between the inlet end 16 and the outlet end 18, as denoted by arrow 24 in FIG. 1.

The inlet end 16 incorporates a generally funnel shaped tip 26. This generally funnel shaped tip 26 may have the effect of reducing the risk of blood clots and also may function to stand the left ventricle open by preventing partial or full collapse of the septum S or left ventricle LV onto the inlet 16. In an alternative embodiment, the inlet end 16 does not incorporate a funnel shaped tip, but rather is cylindrical.

Referring to FIGS. 1 and 2, a filter 28 is positioned within the bore 22 of the inflow cannula 12. The filter 28 is positioned to form a filter or selective barrier across the bore 22. The filter 28 of this embodiment is configured with a pore size to filter or capture thrombus or blood clots passing therethrough prior to the clots entering the blood pump 10.

The tubular body 20 may be made of a biocompatible polymeric material such as: polyurethane (‘PU’); polyetheretherketone (‘PEEK’); or silicone moulding. The tubular body 20 of this embodiment is constructed so as to prevent collapse when under negative pressure but flexible enough so that a surgeon or clinician is able to bend or flex the inflow cannula 12 during implantation.

Referring to FIG. 1, the outflow cannula 14 is connected between the LVAD 10 and the aorta A. Generally, the outflow cannula 14 does not include a filter as only very low levels of thrombogenesis or blood clots occur within the preferred LVAD 10. The outflow cannula 14 is preferably constructed of woven Dacron or velour material for providing a suitable fluid conduit that is capable of experiencing positive pressure as produced by the LVAD 10. The outflow cannula 14 is generally anatomised to the aorta 3 by stitching. Additionally, the outflow cannula 14 may be partially or fully encased within a semi-rigid bend relief (not shown), these bend reliefs are commonly made or constructed of semi-rigid biocompatible polymeric substances. The bend relief may function to prevent kinking or collapse of the outflow cannula 14.

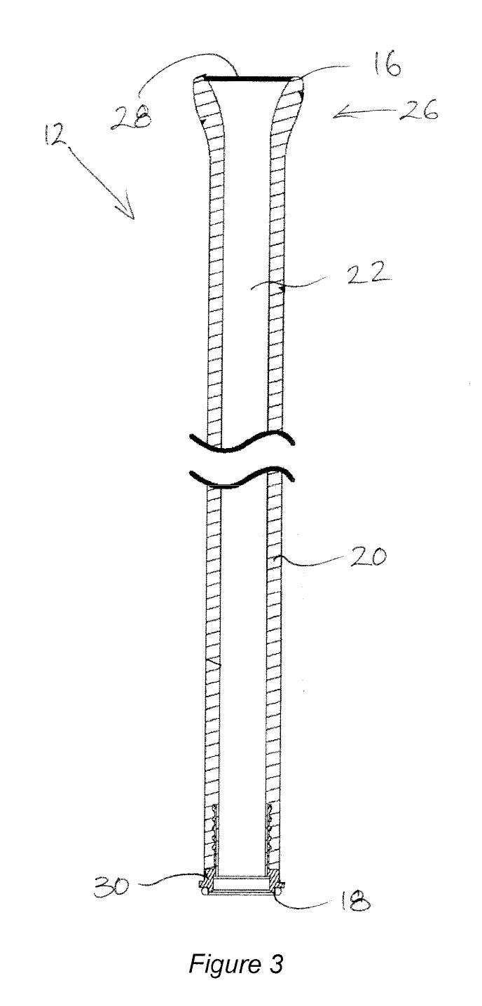

The filter 28 has been described above with reference to FIGS. 1 and 2, where it is located about halfway along the body 20 of the cannula. However, in alternative embodiments, it can be located elsewhere in or on the cannula 12. For example, as illustrated in FIG. 3 where like reference numerals denote like parts, it may be located at the tip 26 of the inlet end 16. As per the previously described embodiment, the inlet end 16 is adapted to be received by the left ventricle LV through the incised hole 17 in the apex of the left ventricle LV. In this embodiment, the filter 28 is positioned so that when in use the filter 28 is inside the left ventricle LV. As the filter 28 is preferably proximal or near to the inlet end 16 of the inflow cannula 12, this may further reduce the risk of blood clots blocking the inflow cannula 12.

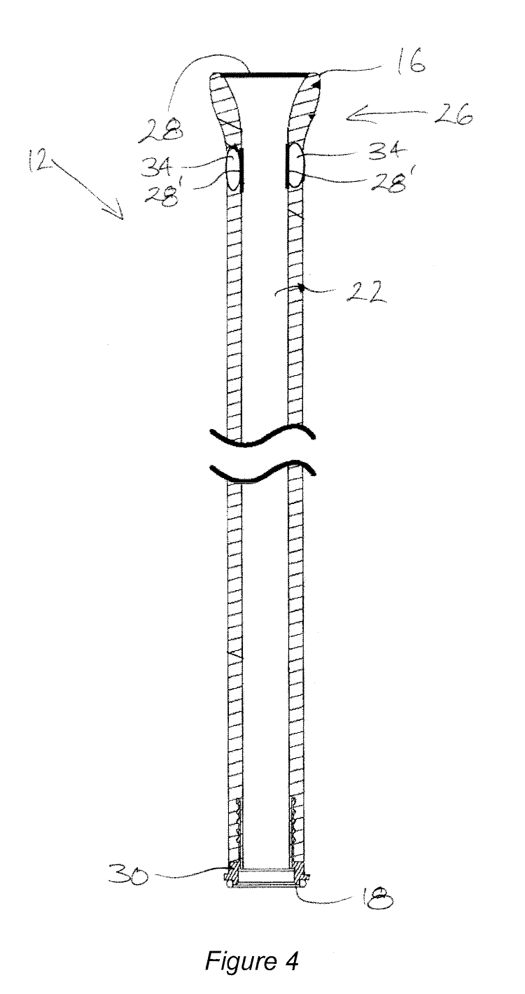

FIG. 4 depicts another embodiment where like reference numerals denote like parts. In this embodiment, the inlet end 16 additionally includes two apertures 34 mounted immediately below the tip 26. When the inflow cannula 12 is implanted within the left ventricle LV, the side apertures 34 are also inserted within the cavity of the left ventricle LV. The side apertures 34 assist the funnel tip 26 in situations where blockage occurs by the blood to have a secondary flow-path. The side apertures may also include a filter 28′ covering each side aperture 34 to allow for the inflowing blood using the side apertures 34 to be filtered also. In an alternative embodiment, there are no filters covering the side apertures 34.

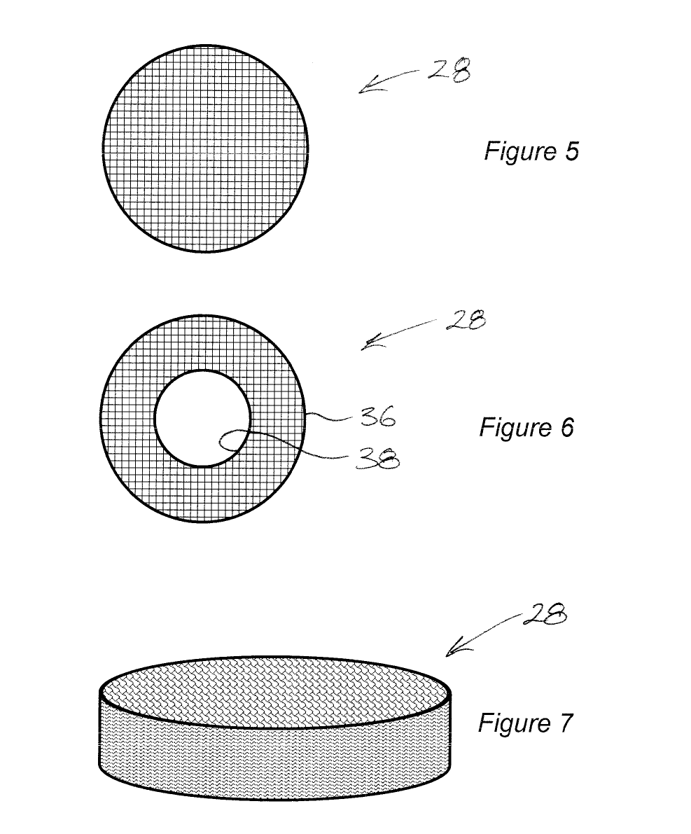

FIG. 5 illustrates the filter 28 used with each of the above described embodiments. In this embodiment, the filter 28 is a biocompatible wire mesh having a two-dimensional matrix structure. The mesh in this embodiment is a titanium alloy, such as Nitinol, which has been coated with a secondary substance chosen from the group including, but not limited to polyurethane, diamond-like carbon, polyetheretherketone and silicone. Alternatively, or in addition thereto, the mesh may also be coated with a substrate that carries an electrical charge that is appropriate to attract and capture blood clots. Also alternatively or in addition thereto, the substrate may be used to carry a pharmacologically active product such as drugs that inhibit or reduce blood clots (e.g. heparin).

FIGS. 6 and 7 illustrate alternative embodiments of the filter 28. In the embodiment illustrated in FIG. 6, the filter area is annular, defined by an outer circumference 36 of the filter 28 and an aperture 38, such that no filtration of blood occurs through the aperture 38. This arrangement helps to ensure some filtering of the blood can occur, while also ensuring or preventing blockage of flow therethrough. When this filter 28 is positioned at the inlet end 16, it may also provide a substrate for tissue growth thereon, thus preventing thrombus associated therewith from entering the LVAD 10.

FIG. 7 illustrates another embodiment of the filter 28, which has a three-dimensional mesh-like filtration structure, and which is illustrated with an exaggerated thickness. The thickness is in the range of about 50 μm to a few millimetres, or preferably 50 μm to 500 μm, or preferably 50 μm to 300 μm, or preferably 120 μm to 220 μm. In this embodiment, the filter is generally cylindrical and porous therethrough. The filter is manufactured from a suitable biocompatible porous structure such as titanium mesh, woven polymeric or titanium filament, or polyester fibre, such as DACRON®.

The pore size of each of the above described filters is small enough to capture at least some blood cots in blood passing therethrough, yet large enough at least to allow individual blood cells to pass therethrough. For example, the pore size of the filter of any of the embodiments ranges from about 20 μm to 500 μm, or preferably 20 μm to 300 μm, or preferably 30 μm to 300 μm, or preferably 50 μm to 300 μm.

In other embodiments, the filter may not be flat, but may be cup-shaped, concave, or similar.

FIG. 8 illustrates another embodiment, where like reference numerals denote like parts, being a heart assist device in the form of an LVAD 10. The filter 28 is upstream of the LVAD 10 to filter at least a portion of blood entering the LVAD 10. In this embodiment, the filter 28 is located at the inlet 19 of the LVAD 10.

The above embodiments have been described with reference to the use of an LVAD. In alternative embodiments, other types of blood pumps can be used, such as right ventricular assist devices, axial flow blood pumps, pulsatile blood pumps, and so on.

As will be understood, unless the context requires or suggests otherwise, features of any one of the above described embodiments may be used in conjunction with another one or more of the above described embodiments.

While the invention has been described in reference to its preferred embodiments, it is to be understood that the words which have been used are words of description rather than limitation and that changes may be made to the invention without departing from its scope as defined by the appended claims.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

A reference herein to a prior art document is not an admission that the document forms part of the common general knowledge in the art in Australia or elsewhere.

1. A cannula for an implantable blood pump, the cannula comprising: a body having a bore extending between an inlet and an outlet for fluid communication therebetween; and a filter disposed in, on or at the body and arranged to filter blood clots from at least a portion of fluid passing through the body.

2. The cannula of claim 1 being an implantable inflow cannula.

3. The cannula of claim 1 wherein the body is tubular.

4. The cannula of claim 1, arranged to be mounted between a patient's left ventricle and an implantable blood pump.

5. The cannula of claim 1, wherein the filter is located at or adjacent the inlet.

6. The cannula of claim 1, wherein the filter is located in or on the body such that all the fluid passing through the body will pass through the filter.

7. The cannula of claim 1 wherein the filter is annular defining an outer screening portion and a central non-screening aperture and is located in or on the body such that some of the fluid passing through the body will pass through the outer screening portion and some of the fluid will pass through the central aperture.

8. The cannula of claim 1, wherein the filter is constructed of a mesh.

9. The cannula of claim 1, wherein the filter is constructed of titanium alloy.

10. The cannula of claim 1, wherein the filter is coated with a biocompatible substrate.

11. The cannula of claim 10, wherein the substrate carries a charge appropriate to attract and capture clots.

12. The cannula of claim 1, wherein the body is constructed of silicone.

13. The cannula of claim 1, wherein the inlet is configured to be inserted within the left ventricle of the patient such that the filter is located on the inlet within the left ventricle.

14. A heart assist device comprising:
a blood pump;
a blood inlet and a blood outlet;
a blood passage from the inlet to the outlet through the pump; and
a filter in cooperation with the blood pump arranged to filter at least a portion of blood passing through the blood pump.

15. The device of claim 14 wherein the filter is located upstream of the pump.

16. The device of claim 14 wherein the filter is arranged to filter all the blood passing through the pump.

17. The device of claim 14, wherein the filter comprises a titanium alloy mesh or matrix.

18. The device of claim 14, wherein the filter is coated with a biocompatible substrate.

19. The device of claim 18, wherein the substrate carries a charge appropriate to attract and capture clots.

20. The device of claim 14, comprising an inflow cannula in fluid communication with the blood inlet.

21. The device of claim 20 wherein the filter is located in the cannula.

22. The device of claim 20 wherein the filter is located at or adjacent an inlet end of the cannula.

23. The device of claim 20, wherein the cannula has two ends, a first said end being connectable to the blood inlet and a second said end being mountable to a patient's left ventricle.

24. The device of claim 14 wherein the filter is located adjacent an inlet of the device.

25. The device of claim 14 wherein the device is a ventricular assist device.

26. The device of claim 14 comprising the cannula of claim 1.

27. A heart assist device filter for filtering blood passing through the device, the device comprising a blood pump having a blood inlet and a blood outlet and a pumping means for pumping blood through the device, wherein the filter is positionable upstream of the pumping means to filter at least a portion of blood entering the device.

28. (canceled)
